(19) United States
(12) Patent Application Publication
(10) Pub. No.: US 2016/0106445 A1
(43) Pub. Date: Apr. 21, 2016

(54) ASSEMBLY FOR LIGAMENT REPAIR AND INCLUDING A CUTTING TOOL

(71) Applicant: Ronald SEKEL, Kogarah, NSW (AU)

(72) Inventor: Ronald SEKEL, Kogarah (AU)

(21) Appl. No.: 14/894,409

(22) PCT Filed: May 28, 2014
(86) PCT No.: PCT/AU2014/000554

§ 371 Date: Nov. 27, 2015

(30) Foreign Application Priority Data
May 28, 2013 (AU) 2013901899

(51) Int. Cl. A61B 17/17; A61B 17/15; A61B 17/16

(52) U.S. Cl. CPC A61B 17/1764; A61B 17/1637; A61B 17/1675; A61B 17/157; A61B 17/1714; A61B 2017/0046

(57) ABSTRACT

A surgical tool, for use in a ligament repair procedure after rupture of the ligament, the tool comprising; a handle having a first end and a second end, the second end capable of receiving and retaining a cutting guide assembly; the cutting guide including an outer wall which defines an internal, space in which there is located a base; a passage defined by the outer wall and base and which allows access for a cutting implement to bone; and at least one formation extending from the cutting guide which locates the tool against bone during cutting; wherein the bone cutting guide, guides the cutting implement through said passage to enable cutting of a bone section which is separated from surrounding bone.

### BACKGROUND

The present invention relates to surgical tools and appliances and more particularly, relates to an assembly for enabling advancement and retraction of bone when cut during a surgical procedure. More particularly the invention relates to a tool which enables the creation of a bone core which is freed from surrounding bone to enable advancement and retraction. The invention further relates to a surgical kit including a tool which allows a surgeon to make a core of bone with or without tendon or ligament attached which can move relative to surrounding bone for altering the distance between a soft tissue anchored to the core during repair of ruptured soft tissues such as ligaments and the like. The invention further relates to a tool which allows repair of a ruptured tendon without using a graft.

### PRIOR ART

Cruciate ligaments occur in the knee of humans and other bipedal animals and in the neck, fingers, and foot. The cruciate ligaments of the knee are the anterior cruciate ligament (ACL) and the posterior cruciate ligament (PCL). These ligaments are two, strong, generally rounded, bands of varying cross sections along the length that extend from the head of the tibia to the intercondyloid notch of the femur. The ACL femoral attachment is lateral and the PCL is medial. Due to a twist or rotation along the length of the ligament the proximal and distal ends are disposed so they cross each other like the limbs of an X. In other words their respective anchorages are in a sense out of phase. This makes the cruciate ligament a complex structure with a complex anatomical geometry. The ACL and PCL remain distinct throughout and each has its own partial synovial sheath. Relative to the femur, the ACL keeps the tibia from slipping forward and the PCL keeps the tibia from slipping backward. It is a critical ligament for posterior/anterior knee stability. ACL injuries are among the most common knee injuries suffered by sports people. A ruptured ACL can occur by a movement as benign as a sudden change of direction or in more traumatic impact such as falling awkwardly in a tackle and abnormal knee extensions. Anatomical characteristics of a particular individual may predispose one individual to an ACL rupture more so than another individual engaged in the same knee extension, trauma, impact etc. After an ACL reconstruction patients have varying degrees of successful results in that some can return to high level sports whereas others must be content with retirement from sports. Prognosis post surgery can depend upon the quality of the ligament repair and the method of repair or reconstruction used in surgical treatment. After surgery patients are concerned about the risk of repeating the injury particularly if attempting a return to high level sports, in which the knee is loaded and stressed. The incidence of Anterior Cruciate Ligament (ACL) tears requiring surgery is about 50 per 100,000 in the general population in Australia. Males are more likely than females to rupture an ACL as a result of sports injuries. Prognostic factors in determining outcomes of ACL repairs include age, gender, timing of surgeries and other concomitant injuries such as cartilage and meniscal injuries.

One of the difficulties faced by surgeons in ACL or PCL repair is the loss of natural cruciate ligament tissue when ruptured. Each individual has a cruciate of finite length. If ruptured, the tissue at opposing ends at the rupture becomes frayed. In a case where the elected surgical method requires the frayed ends to be sewn together the loss of tissue length can inhibit an optimal result and inevitably results in failure of the ligament to heal compromising knee stability and the development of instability. In cases where the cruciate ligament cannot be sewn back together grafts can be employed as a substitute for the ruptured cruciate ligament.

A knee reconstruction can involve repair of the cruciate ligament or use of grafts. A graft for example may be harvested from the patella tendon in the knee—a bone patellar tendon bone autograft,—or from the hamstring—autogenous hamstring tendons. Surgical outcomes can be dependent upon the type of graft used for the repair, age and gender of the patient, durability of the graft. In high level sports a patellar tendon graft may be the preferred selection but a hamstring graft can work just as well in male or female patients. In a graft repair the ends of the graft must be anchored to femoral and tibial bone to simulate as far as is anatomically possible the natural anchorage of the cruciate ligament to bone. Screws and rods are used to effect an anchorage of the new graft but a graft is not usually as strong as the natural anatomy of the cruciate ligament. As the cruciate ligament has a complex structure and a unique geometry a tendon harvested from another site although very often effective in a repair is not ideal.

An ideal repair method is to re join if possible the anterior cruciate ligament in the knee if there is sufficient length of the ligament left after the rupture. It can be very difficult to rejoin ruptured cruciate ends due to the loss of length on rupture indicated earlier. If possible, the ends are stitched together but they must be stretched to a tension beyond the cruciate ligament's normal rest tension. Post operative physiotherapy is then used in an attempt to regain as much of the original knee function and range of movement existing before the ACL rupture.

The current gold standard treatment option for mid-substance ACL tear is reconstruction with patellar or hamstring tendon autografts. The operation generally yields good results in many patients, although it carries an approximately 3% revision rate at 5 years. The reported rates of patients returning to pre-injury activity levels range widely from 26% to 90%. That means 10-74% patients did not have excellent results after the operation, indicating that there is still room for improvements in regards to ACL repair procedures.

There are two intrinsic surgical difficulties encountered with the current standard surgical treatment of ACL rupture. Firstly, the human ACL is not a simple cylindrical structure but has a complex anatomy consisting of at least 2 rotary bundles which are difficult to replicate. Secondly, the ACL tibial and especially femoral attachment sites have an ellipsoid rather than a circular footprint which has not been successfully reproduced by the single, double or four stranded ACL graft repairs that are currently used.

Primary repair of the ACL trialled in the past encountered the problem of non-union and subsequent failure of the ligament. The reason that many intra articular tissues fail to heal has been attributed to the lack of blood supply and fibroblastic proliferation. However, histological studies of ruptured ACL revealed that the proliferation of fibroblasts and angiogenesis does occur in ruptured ACLs and therefore ACL should have the healing potential similar to other ligamentous tissues.

There are histological reasons which may explain why repaired ligaments fail.
1. The expression of actin-containing smooth muscle cells in the synovial tissue results in the retraction and the formation of a gap between the ACL ends which prevents the healing, process;
2. The subsequent formation of synovial tissue over the discontinuous ruptured ACL ends has been postulated to further impede the ACL healing process. It is hypothesised that the overlay of the two ruptured ACL ends could potentially overcome the problem of the gap formation and non-union in primary ACL repair.

In mid-substance Achilles tendon ruptures, a primary repair yields excellent results as the frayed ends are sutured together in an overlaying manner. A significant outcome-modifying measure in the primary repair of an Achilles tendon is the ability to overlay the two ends by plantar-flexing the ankle joint. The same significant outcome-modifying measure has not been possible in the past with the primary repair of the ACL as the frayed ends could not be overlayed due to loss of length from rupture.

There is an on going need to constantly explore ways to improve the apparatus and equipment for Cruciate and other ligament repairs and to increase the success rate of surgical repair of ligaments.

### INVENTION

With this in mind the present invention provides a surgical assembly including a tool which allows advancement and retraction of a bone core with or without ligament or tendon attached during a surgical procedure for repair of a ligament or tendon. More particularly the invention relates to a tool which is capable of drilling out a bone core from surrounding bone to enable advancement and retraction of the core so that the distance between ends of a soft tissue structure such as a ligament can be adjusted to allow either abutment or overlap of ends during repair. The invention further provides an assembly which includes a tool which allows repair of a ruptured tendon without using a graft.

Outlined broadly below are embodiments and features of the invention to enable the invention to be better understood, and in order that the present contribution to and improvement over the current the art may be better appreciated. There are, of course, additional features of the invention that will be described hereinafter and which will form the subject matter of the claims.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways in various anatomical sites including in veterinary applications. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other variations on the tool assembly, structures, methods and systems for carrying out the purposes of the present invention.

It is therefore an object of the present invention to provide a new and improved tool which removes the practical disadvantages encountered in surgical repair of an ACL and other cruciate ligaments. It is a further object of the present invention to provide a new and improved tool assembly for the above purpose which is of a durable and reliable construction.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

In its broadest form the present invention comprises:
an assembly for use in a ligament reconstruction procedure after rupture of the ligament, the assembly comprising a guide element which has a first end which in use engages a bone surface, a through passage and a second end;
a bone cutting member insertable in said through passage in the guide element;
a pilot wire insertable in a through passage in said cutting member;
a drill bit having a first end which engages said cutting member and a second end which is retained by a drill; wherein the cutting member is capable of advancing and retracting inside said guide element to create a moveable cylinder of cut bone.

In another broadest form the present invention comprises:
a surgical tool for use in a ligament reconstruction procedure after rupture of the ligament, the tool co operating with a guide assembly to enable separation of a cylindrical piece of bone from surrounding bone such that the piece of bone is free to move relative to the surrounding bone;
the tool comprising a first end having a cutting profile and a second end which is operatively connected to a drill to enable rotation of the cutting profile;
the guide assembly including a guide member and a pilot wire which is insertable in the bone to determine a direction for the cutting member.

In another broad form the present invention comprises;
an assembly for use in the re attachment of a ruptured ligament, the assembly comprising;
a cutting tool operable by a power drill and having a first end terminating in a cutting profile and a second end attachable to the power drill, the cutting tool capable of creating by operation of the cutting profile a core of bone to which is attached part of a ligament requiring repair,
an instrument which receives a rod capable of entering a through passage in the instrument; the rod having a leading end which is capable of entering the cutting tool to engage the bone core to allow advancement and retraction of the bone core.
a surgical tool for use in a ligament reconstruction procedure after rupture of the ligament, the tool co operating with a guide assembly to enable separation of a cylindrical piece of bone from surrounding bone such that the piece of bone is free to move relative to the surrounding bone;
the tool comprising a first end having a cutting profile and a second end which is operatively connected to a drill to enable rotation of the cutting profile;
the guide assembly including a guide member and a pilot wire which is insertable in the bone to determine a direction for the cutting member.

The tool is adapted for cutting bone which retains part of a ligament. The inventive concept adopted by the assembly described herein is adaptable for the repair reattachment of a variety of ligaments when shortened on rupture.

The guide member has a first end which in use engages a bone surface, a through passage and a second end. The bone cutting member is insertable in a through passage in the guide member. The pilot wire is insertable in a through passage in said cutting member. A drill bit has a first end which engages the cutting member and a second end which is retained by a drill; wherein the cutting member is capable of advancing and retracting inside said guide element to create a moveable cylinder of cut bone.

The assembly further comprises an extraction device which engages the cutting member to enable withdrawal of the cutting member.

In another broad form the present invention comprises:
a surgical tool for use in a ligament repair procedure after rupture of the ligament, the tool comprising:
a handle having a first end and a second end, the second end capable of receiving and retaining a cutting guide assembly;
the cuttings guide including an outer wall which defines an internal space in which there is located a base;
a passage defined by the outer wall and base and which allows access for a cutting implement to bone; and at least one formation extending from the cutting guide which locates the tool against bone during cutting;
wherein the bone cutting guide, guides the cutting implement through said passage to enable cutting of a bone section which is separated from surrounding bone.

The cutting guide can form a variety of pristine shapes including cylindrical trapezoidalsections of bone isolated from a bone site. Preferably the section of bone has a ligament attached to one face. According to one embodiment the ligament is the cruciate ligament.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1a shows an anterior elevation view of a knee joint.

FIG. 1b shows the knee joint of FIG. 1a enlarged to show the anterior cruciate ligament.

FIG. 2 shows the knee joint of FIG. 1b with a ruptured cruciate ligament and an assembly including a power drill operated coring tool for creating a displaceable core in tibial bone at the tibial end of the cruciate ligament.

FIG. 3 shows the knee joint of FIG. 1b with the core in tibial bone at the tibial end of the cruciate ligament advanced so the severed ends of the cruciate ligament are re engaged.

FIG. 4 shows a schematic elevation view of a core made in tibial bone in isolation from the knee joint.

FIG. 5 shows the core of FIG. 4 with ACL part axially advanced towards an opposing part of ACL to close a gap.

FIG. 6 shows an exploded view of a drill and cutting tool.

FIG. 7 shows the cutting tool of FIG. 6.

FIG. 8 shows an instrument used for advancing and retracting the bone core and relationship with the cutting tool,

FIG. 9 shows a jig assembly which allows a surgeon to establish an accurate drilling axis prior to drilling out core.

FIG. 10 shows a perspective view of a tool according to one embodiment including a handle and cutting guide.

FIG. 11 shows a first side elevation view of the tool of FIG. 10.

FIG. 12 shows a first side elevation view of the tool of FIG. 10.

FIG. 13 shows an enlarged view of the working end of the guide of FIG. 10 engaging a bone section connected to a ruptured end of a cruciate ligament

FIG. 14 shows a schematic view of a bone section cut from tibial bone and advanced to facilitate overlap of ruptured ends of a cruciate ligament.

FIG. 15 shows a perspective view of a tool according to an alternative embodiment including a handle and cutting guide.

FIG. 16 shows a first side elevation view of the tool of FIG. 15.

FIG. 17 shows a first side elevation view of the tool of FIG. 15.

FIG. 18 shows an enlarged view of the working end of the guide of FIG. 15 engaging a bone section connected to a ruptured end of a cruciate ligament.

FIG. 19 shows one embodiment of the geometry, taper angle and dimensions of a cut bone section.

### DETAILED DESCRIPTION

The present invention will now be described in more detail according to a preferred embodiment but non limiting embodiment and with reference to the accompanying illustrations. The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description.

One of the difficulties faced by surgeons in ACL or PCL repair is the loss of natural cruciate ligament tissue when ruptured. Each individual has a cruciate of finite length. If ruptured, the tissue at opposing ends of the rupture becomes frayed. In a case where surgical treatment requires the frayed ends to be sewn together the loss of tissue length can inhibit an optimal result and inevitably results in failure of the ligament to heal which compromises joint stability leaving the joint unstable. In cases where the cruciate ligament cannot be sewn back together grafts can be employed as a substitute for the ruptured cruciate ligament.

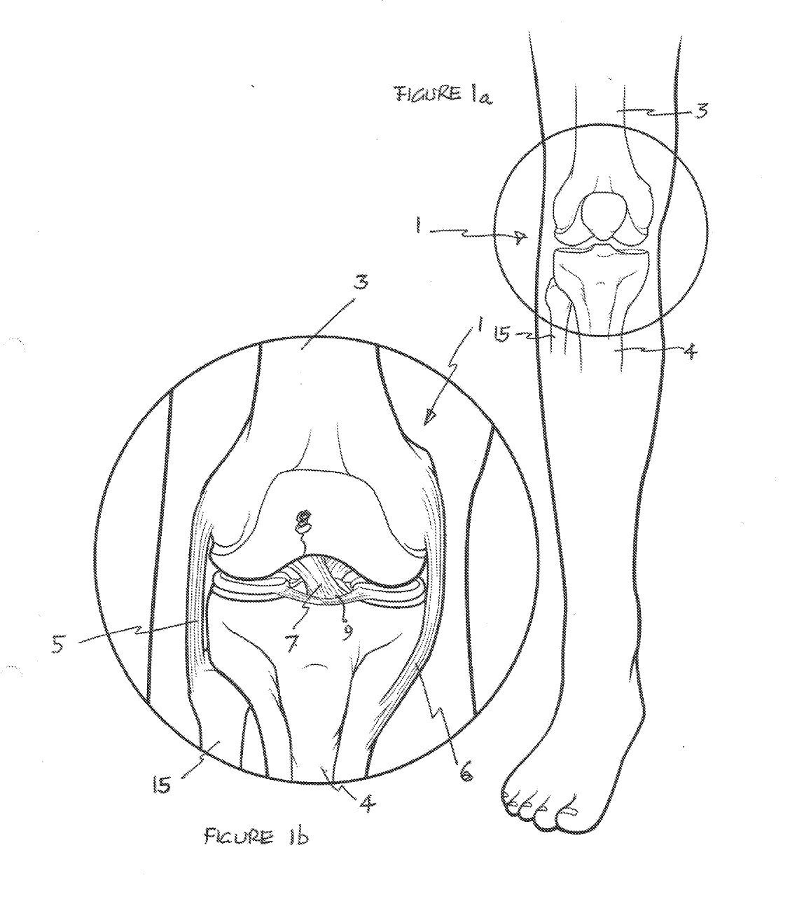

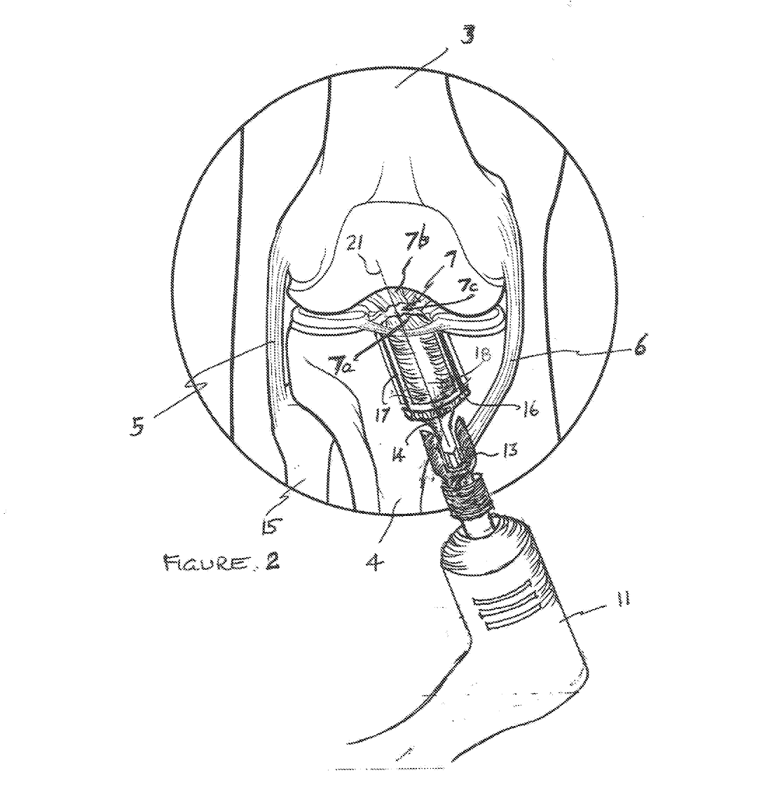

Referring to FIGS. 1a and b there is shown an anterior view of a typical knee joint 1. Knee joint 1 is a schematic anatomical view of a right knee comprising a femur 3, tibia 4 and fibula 15. Joint 1 includes lateral collateral ligament 5 which connects femur 3 to tibia 4 and medial collateral ligament 6 which engages the femur 3 and tibia 4. The present invention will be described with reference to its application to the anterior cruciate ligament (ACL) 7 which is connected at one end 8 to the femur 3 and at the other end 9 to tibia 4.

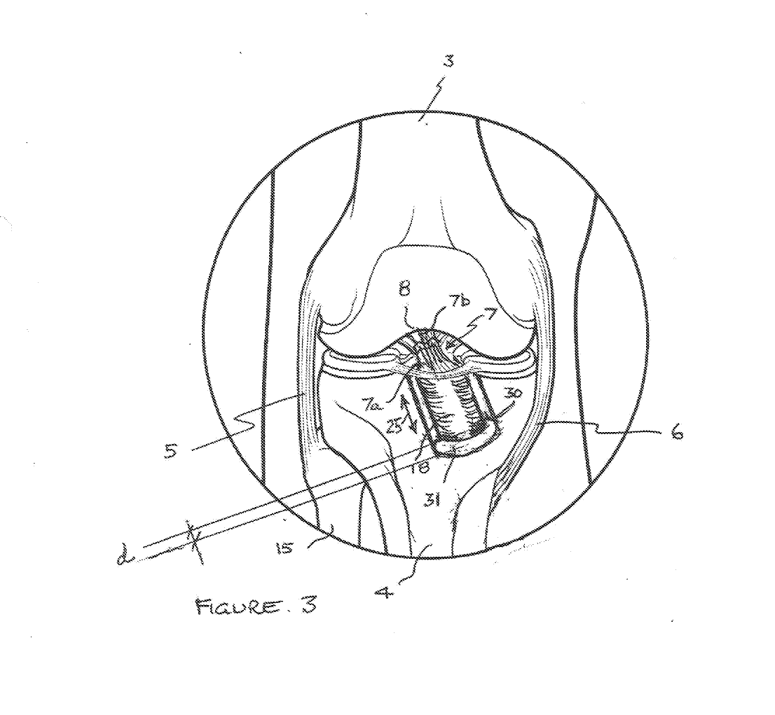

FIG. 2 shows the knee joint of FIG. 1b with corresponding numbering. ACL 7 is shown as ruptured into tibial part 7a and femoral part 7b leaving a gap 7c. Assembly 10 is used in the repair of ACL 7a and 7b and comprises a drill 11 including an adjusting member 12 which operates chuck 13. Chuck 13 retains a bit 14 comprising a base 16 which retains a coring tool 17 which allows a surgeon to drill a core 18 of tibial bone 4. Bit 14 and coring tool 17 are hollow to receive a pilot wire to initially establish an accurate path to the location of the ligament part 7a via the tibia. Core 18 when fully formed is free to advance and retract axially relative to tibia 4. This allows the surgeon freedom to advance core 18 until part 7a of ACL 7 engages part 7b to close gap 7c. One of the problems faced by surgeons when attempting a knee reconstruction by re attaching the cruciate is the gap 7c between frayed ends 7a and 7b. This gap 7c represents a shortening of the ACL 7 which poses difficulty in connecting the ends 7a and 7b. The assembly according to the present invention described herein provides a method for drawing ends 7a and 7b of the ACL 7 closer together to thereby enable more ACL length for overlap and re attachment without having to stitch while the ACL would otherwise be in relatively high tension tending to pull the ends apart. FIG. 2 shows the core 18 prior to advancing tibial part 7a towards part 7b.

FIG. 3 shows with corresponding numbering the knee joint of FIG. 1b with the core 18 in tibial bone 4 at the tibial end of the cruciate ligament part 7a advanced so the severed ends 7a and 7b overlap to allow reattachment thereby closing gap 7c (FIG. 2). By using the appropriate instruments, a surgeon can achieve the advancement of conical core 18, overlay end 7a-to-end 7b followed by suturing and then re-tensioning of the repaired ACL 7 by retracting core 18 and reattaching it to tibial bone 4. One of the ways this may be achieved is by use of an anchorage rod (not shown) disposed transverse relative to the core 18. The new procedure allowed by the assembly 10 also eliminates the morbidity associated with the use of autografts in standard ACL reconstruction (ACLR) techniques (using bone-patellar-bone and hamstring harvesting techniques). As shown in FIG. 3 core 18 advances axially in the direction of arrow 25 once coring tool 17 has created gap 30. Opening 31 allows a surgeon access to the core 18 so it can be advanced and retracted as required. Once ends 7 and 7b of ligament 7 are reattached, tension is re applied to ligament 7 to allows core 18 to be retracted back to as near as possible to its original location in the tibia 4. As ligament 7 is in that case under tension it will tend to pull core 18 towards the ligament due to the tension in the ligament. To resist this tendency, a transverse fixation or other suitable means for anchoring core 18 is adopted. Core 18 can advance at least as far as distance d which will preferably fall within the range of 2-10 mm,

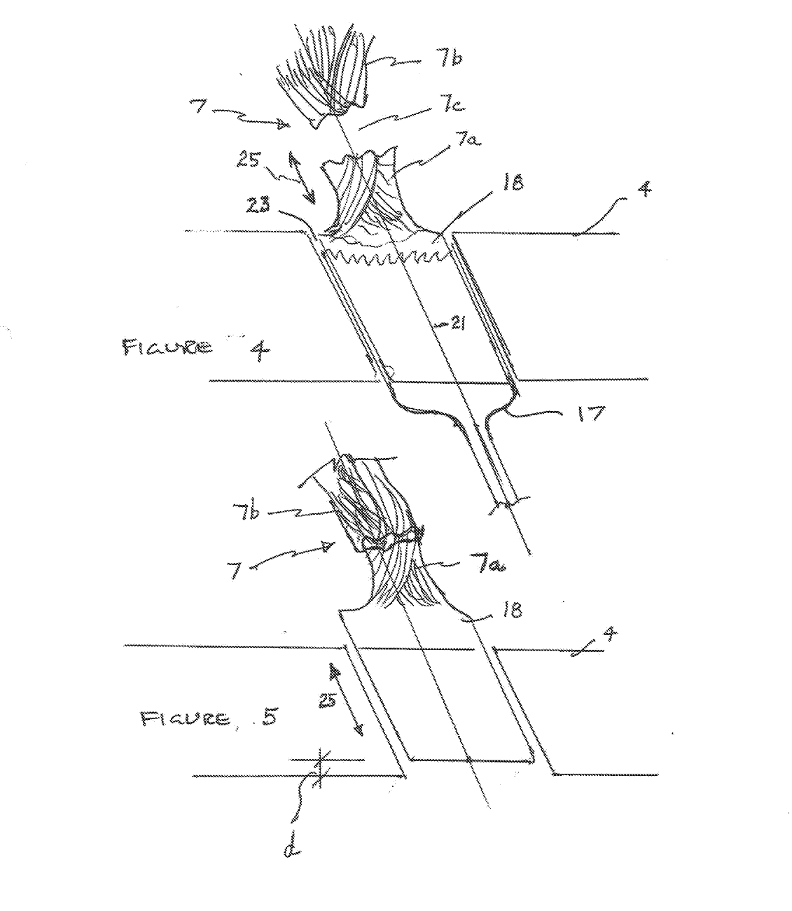

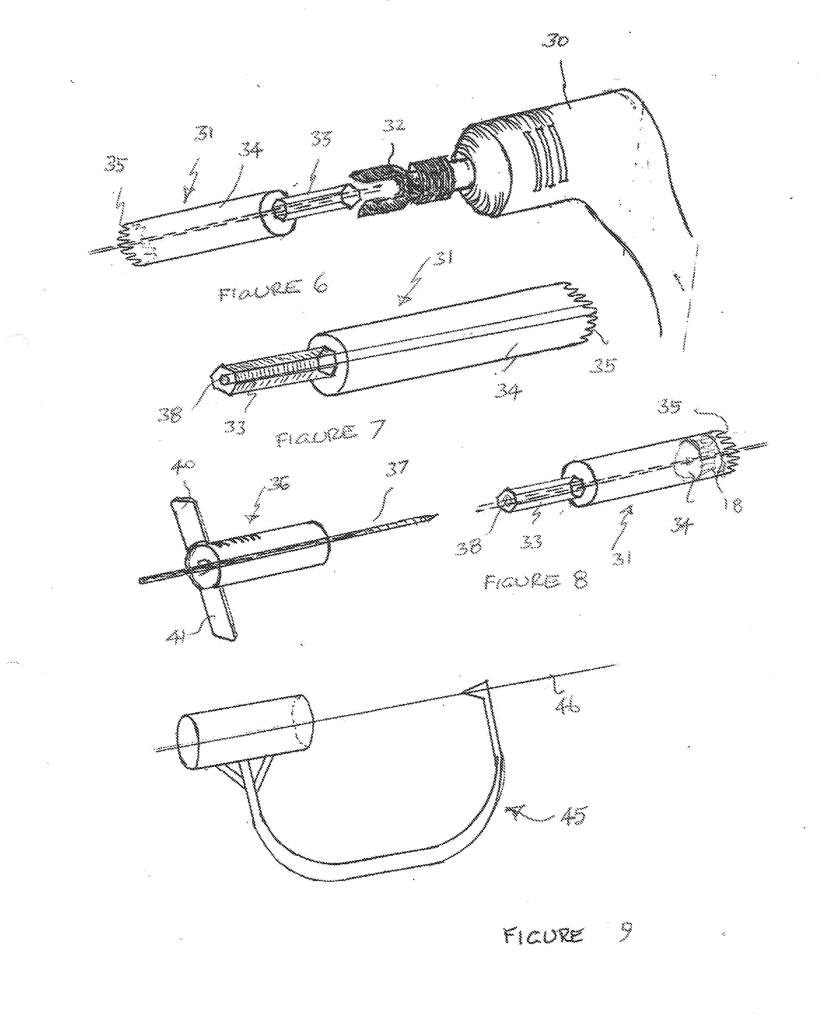

FIG. 4 shows a schematic elevation view of a core 18 made in tibial bone 4 in isolation from knee joint 1. The tool 17 is shown creating the core 18 by generating a circumferential gap 23 which isolates core 18 from tibia 4. Core 18 has extending therefrom a severed part of a cruciate ligament 7a which opposes corresponding end 7b of femoral end of ACL 7. As ACL 7 in FIG. 5 is shown ruptured a gap 7c is left requiring closure for repair. Core 18 in this view is shown as cylindrical but it will be appreciated that other shapes are contemplated such as but not limited to wedge shaped and trapezoidal

FIG. 5 shows the core 18 of FIG. 5 with ACL part 7a is axially advanced towards opposing part of ACL 7b to close gap 7c. Advancement of the core 18 in the direction of arrow 25 is enabled by the core drill bit 17 which separates core 18 from tibial bone. Core drill bit 17 may be a cylindrical crown bit or any attachment or bit capable of drilling a cylinder of bone. Core 18 is free to advance and retract as required by the surgeon over a range of 0-10 mm. The distance of axial movement of core 18 required is dictated by the size of gap 7c plus any overlap required to facilitate stitching/reconnection of the ACL parts 7a and 7b.

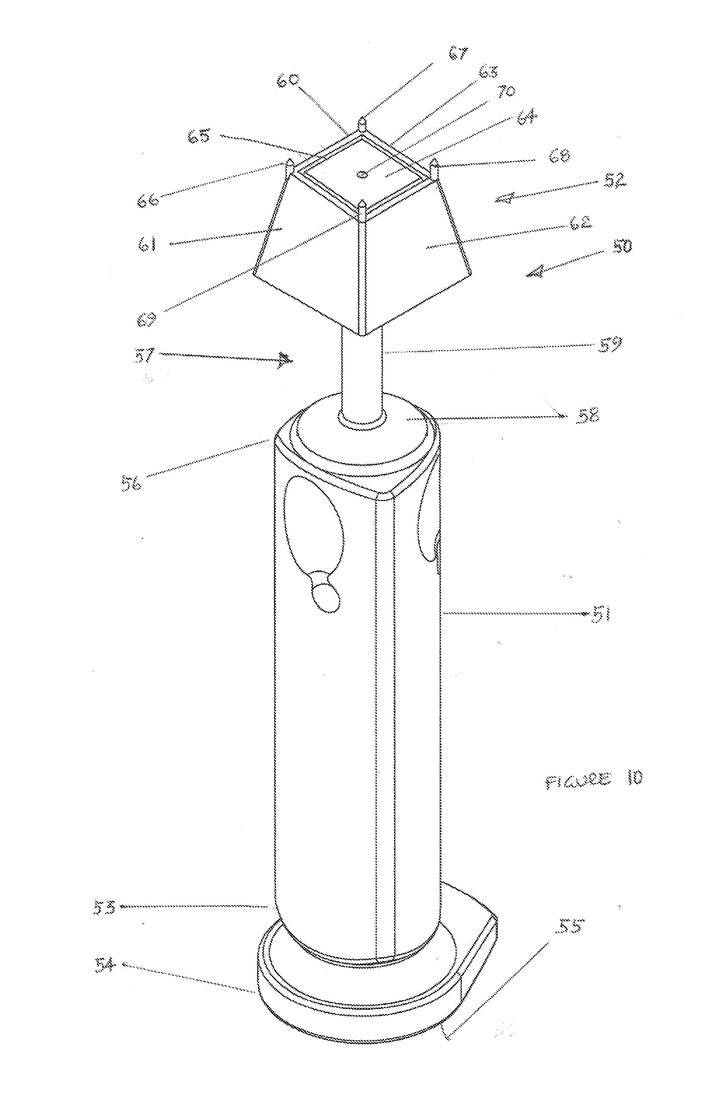

FIG. 6 shows an exploded view of a drill 30 and cutting tool 31. Drill 30 includes a conventional chuck 32 which receives via stem 33 cutting tool 31.Cutting tool 31 comprises a cylindrical body 34 which terminates in a cutting profile 35. FIG. 7 shows an enlarged view of the cutting tool 31 of FIG. 6. A preferred diameter for cutting tool 31 is approximately 9 mm but it will be appreciated that other diameters may be selected according to requirements.

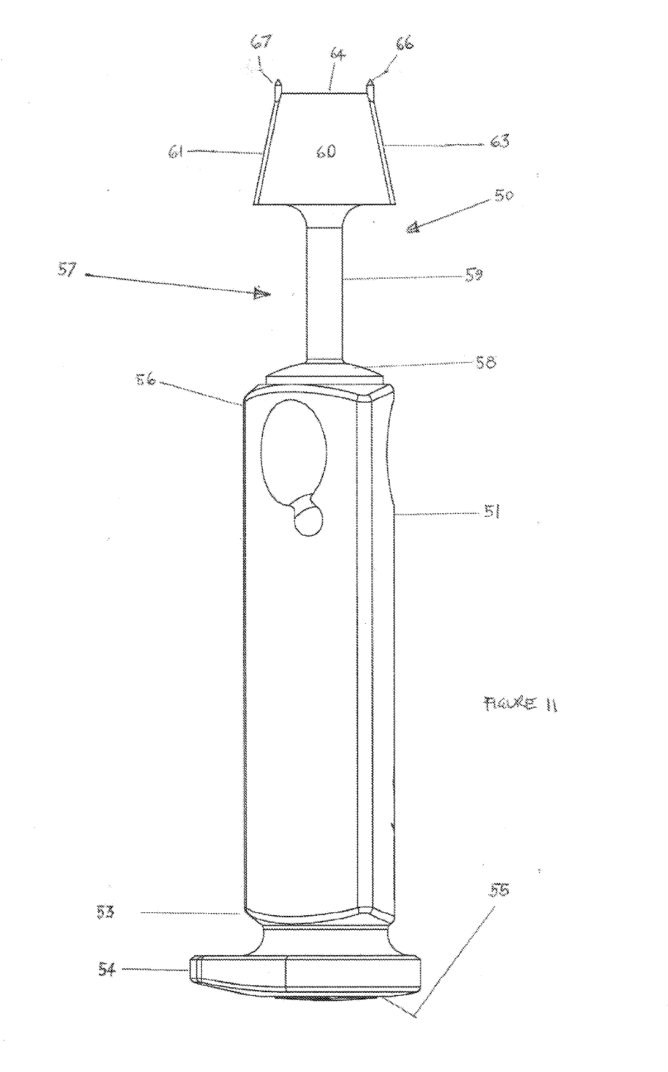

FIG. 8 shows au instrument 36 used for advancing and retracting the bone core 18 (see FIGS. 2-3), and relationship with the cutting tool 31. Since cutting tool 31 is hollow, rod 37 associated with instrument 36 can be advanced through opening 38 of cutting tool 31 until end 39 engages bone core 18. Instrument 36 includes flanges 40 and 41 which facilitate advancing and retracting bone core 18. The surgeon is able to push or pull on bone core 18. Core 18 is advanced to close gap 7c as shown in FIG. 3 and using instrument 36 the bone core can be pulled anteriorly for repositioning and anchorage to the tibia. Instrument 36 includes incremental gradations which allow selection of a distance to advance core 18. According to one embodiment the incremental adjustment for movement of bone core 18 can be set with in the range of 0.5 mm-10 mm. A sleeve guard may be used to surround the cutting tool and provide a directional guide. The cutting tool according to one embodiment may be in two parts such that a proximal end can be detached to allow access to the inside of the cutting tool. Approximate sizes for the cutting tool diameters are in the region of 10 mm-14 mm. The cutting tool can be removed using a slap hammer or the like. Alternatively the cutting tool Can be removed by screwing. A gap between tibial bone and the bone core 4 will be in the region of 0.2 mm-1 mm but it will be appreciated that this size is variable according to instrument design.

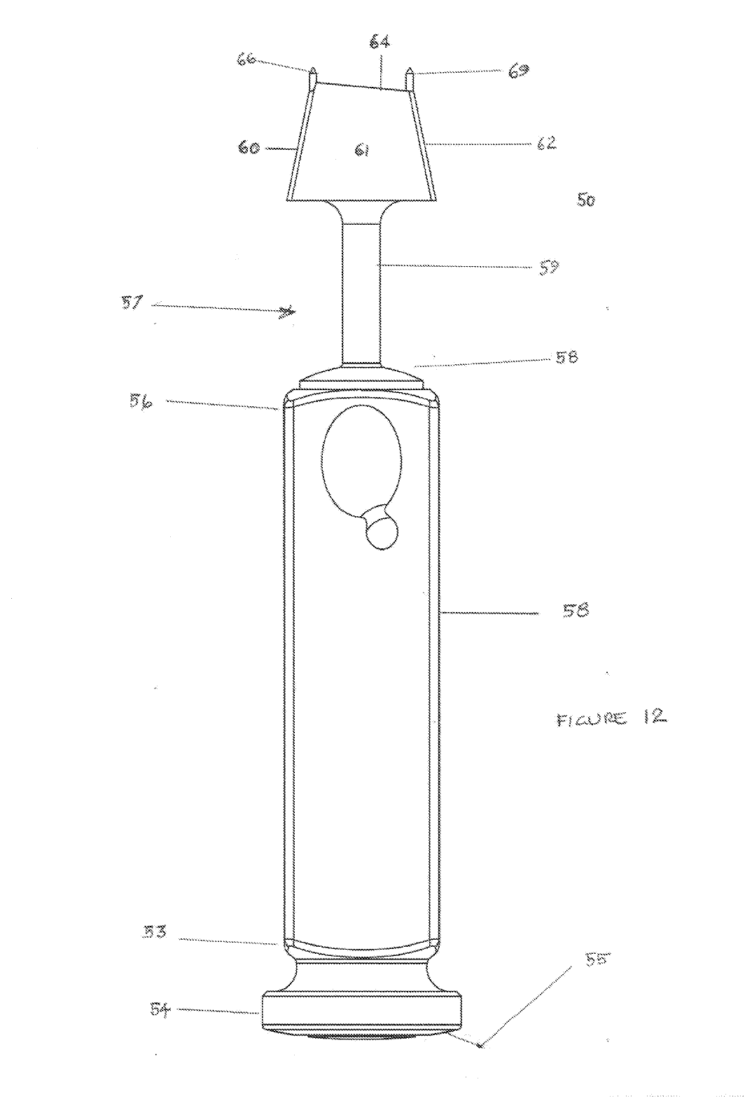

FIG. 9 shows a jig assembly 45 which allows a surgeon to establish an accurate drilling axis 46 prior to drilling out core. The jig holds the cutting tool at the correct angle of entry or enables setting of a drilling path using a pilot wire.

One method of performing an ACL repair using the assembly and associated jig 45 is described below. A minimally invasive mini medial arthrotomy incision is made approximately 3 cm in size. An ACL alignment jig is used to guide and drill a say 1.5 mm Kirschner wire 21 into the centre of the proposed tibial bone core 18 exiting the centre of the distal ACL tibial attachment 7a. A circular crown saw 31 is fitted centrally over the Kirschner wire 21 by using a centrally cannulated alignment jig 45 (see FIG. 9). A tailor made slotted crown saw alignment guard is then fitted over the crown saw to secure the sawing direction with teethed edges 35. The bone core 18 containing the distal ACL segment 7a is cut in the tibia with the circular crown saw guided by the centrally cannulated alignment jig 45. The diameter of the tibial bone core cut by the crown saw will be say 12, 14 or 16 mm depending on patient size. This step can be performed by computer guided tracking.

Using a small curved flexible chisel, the final cortex of the tibial bone tunnel is broken through via access from the bone tunnel. A straight needle is used to pass a suture up the pre-drilled tunnel exiting at the ACL tibial attachment 7a site. The suture is removed from the needle and passed through the ACL distal stump and then repassed down the tunnel using the straight needle. A clip is then applied to the two ends 7a and 7b.

Using a round punch the tibial bone core is advanced up its tunnel for approximately 5 mm (guided by a slotted alignment guard) to allow overlap and end 7a-to-end 7b repair of the central body ACL rupture. A bone anchor is then inserted over each side of the proximal attachment of the ACL to the femur. Using a say Bunnell type suture, the two ends of the torn ACL 7a and 7b will be repaired via a medial arthrotomy. This may he performed using an arthroscopic procedure.

The bone core is then retrieved down its tunnel by either
a.) traction on the suture which is tied firmly over the tibia with an endobutton or similar apparatus; or
b.) pushing the tibial bone core down via access from the medial arthrotomy, leaving mild tension only at the repair site. The bone core is anchored onto the tibia with cross wires. The wounds are closed in the standard manner using sutures. The knee is immobilized in a hinge brace with the same postoperative program as that used for contemporary anterior cruciate repair (ACLR).

Using the core advancement technique the tibial bone core containing the distal ACL stump can be mobilised 3 to 5 mm proximally. An overlay repair of the two ruptured ACL ends 7a and 7b can be achieved The tibial bone tunnel containing the distal ACL stump can be fixed distally. The repaired ACL can be kept intact throughout the surgical procedure.

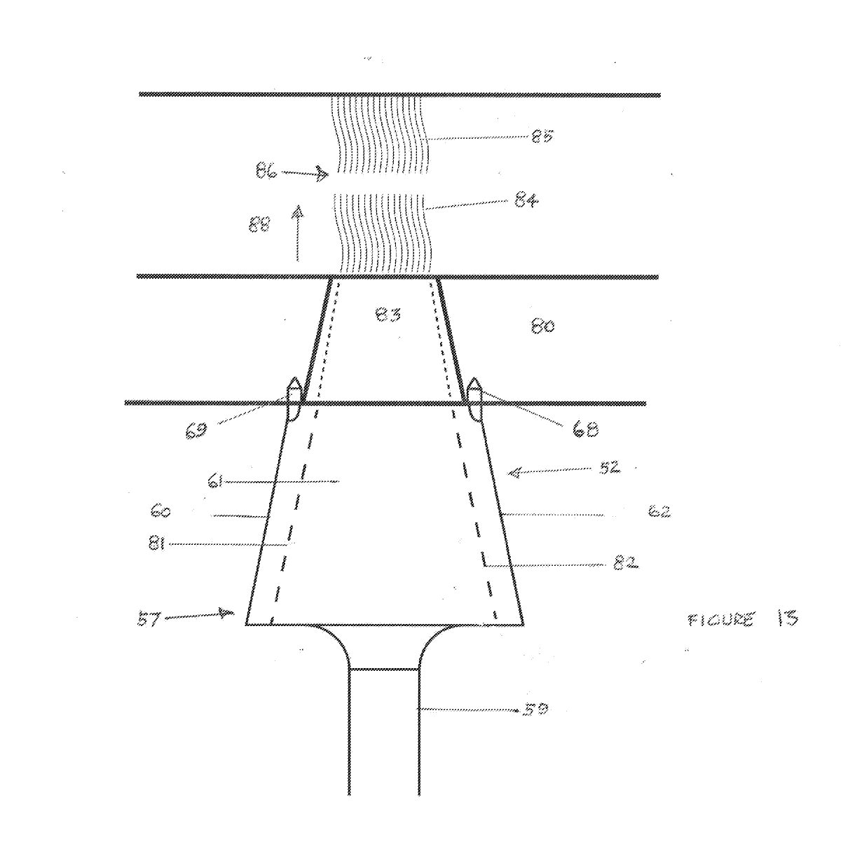

FIG. 10 shows a perspective view of a tool 50 according to one embodiment including a handle 51 and cutting guide 52. Handle 51 has a first end 53 which provides a platform 54 which allows support of the tool 50 and provides an under surface 55 which allows hammer impact when tool 50 is located in position. Handle 51 includes a second end 56 which detachably receives and retains cutting guide attachment 57. Guide element 57 includes a connector 58 which engages handle 51 and is detachable therefrom. Connecting stem 59 terminates at guide 52. Guide 52 according to the embodiment shown includes angled walls 60, 61, 62 and 63 which define in conjunction with platform base 64 an internal passage 65 which is angled in a similar manner to the walls 60, 61, 62 and 63. Extending from cutting guide 52, are four locating posts 66, 67, 68 and 69 which in use are hammered by a surgeon into tibial bone to secure guide 52 and prevent movement when bone cutting through passage 65. This anchors cutting guide attachment 57 against a bone surface to be cut and prevents once in situ, unwanted rotation of the tool 50 and cutting guide 52 during cutting and drilling. Since walls 60, 61, 62 and 63 are splayed inwards in a direction away from handle 51, this provides guidance for a saw used in cutting bone to create a bone section. Guide 52 further comprises an opening 70 which receives a guide wire not shown which aligns with a base footprint of a distal part of a ruptured cruciate ligament. Tool 50 allows a surgeon to create a wedge or trapezoidal shaped bone section which is free to move axially within a passage 100 created by the cutting tool. Various shapes are feasible to adopt for the surgical method of cruciate repair but ideally the bone section will have a narrow width at its proximal end than its distal end. The shapes can be selected according to the selection of the geometry of the cutting guide. This allows the bone section move a short distance in the passage formed and eventually engage tibial bone to resist further movement. The movement allows a distal portion of a ruptured ligament to overlap with a proximal part of that same ruptured ligament thereby facilitating stitching of the opposing ligament parts.

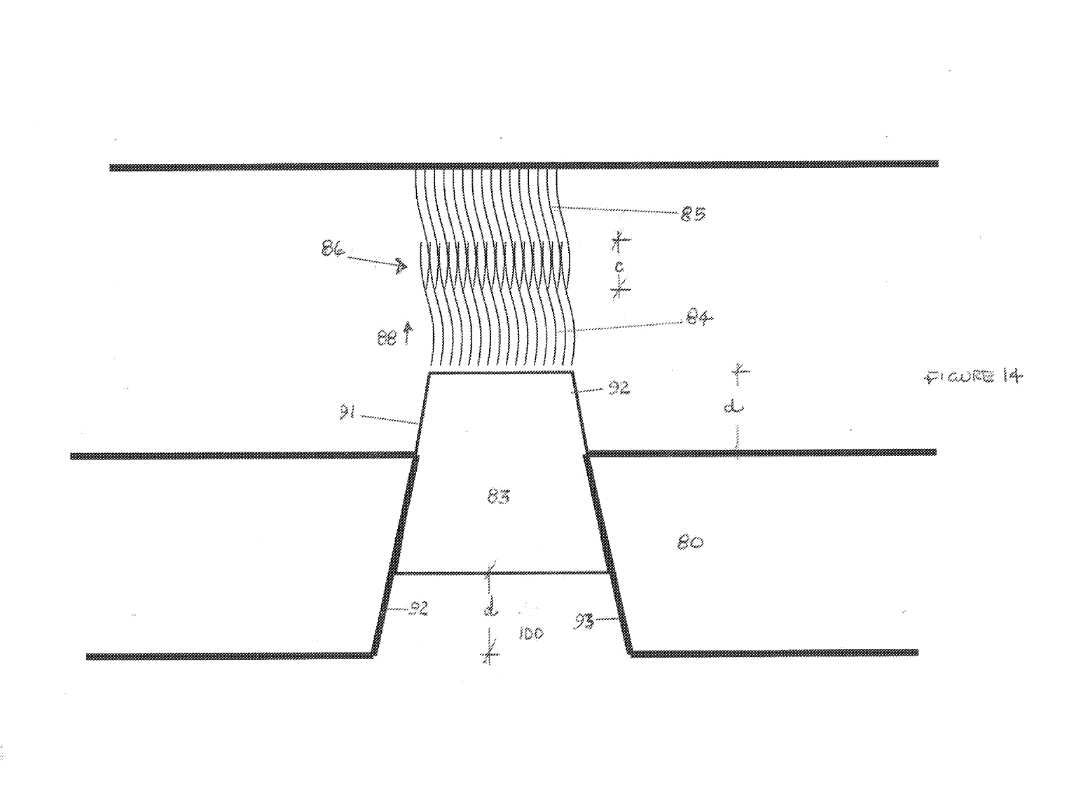

FIG. 11 shows with corresponding numbering a first side elevation view of the tool of FIG. 10. FIG. 12 shows with corresponding numbering a first side elevation view of the tool of FIG. 10.

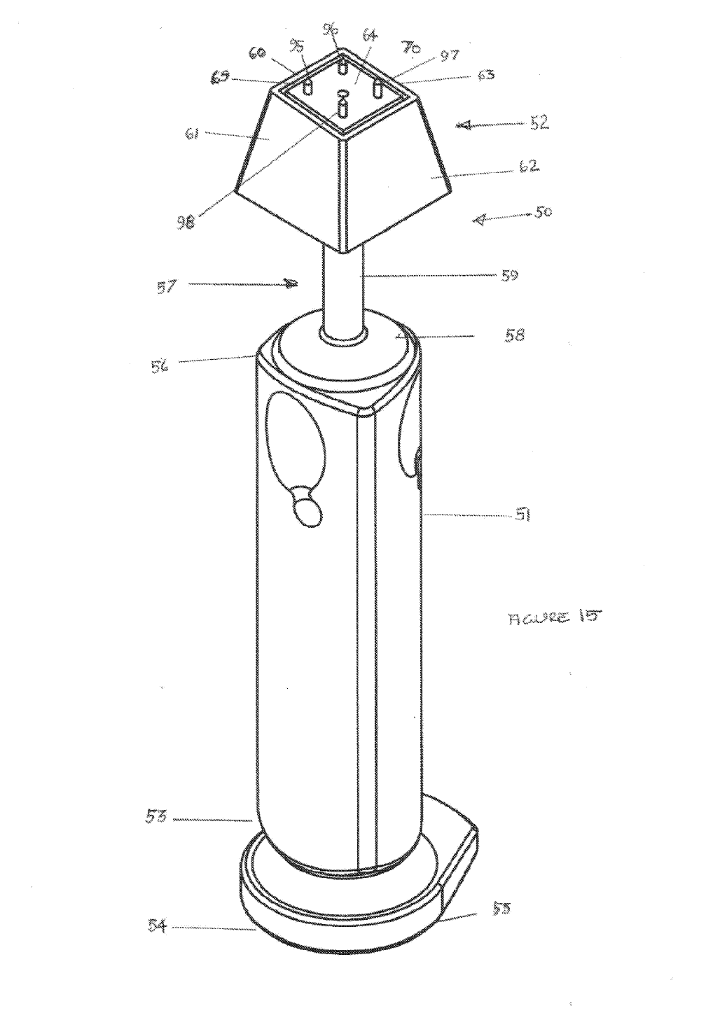

FIG. 13 shows with corresponding numbering an enlarged view of the working end of the cuffing guide 52 of FIG. 10 engaging a schematic view of tibial bone 80 via posts 68 and 69. Dotted lines 81 and 82 indicate a passage through which a saw blade may be passed advancing towards bone section 83. Extending from bone section 83 is a distal part 84 of a ruptured cruciate ligament 86 which opposes a proximal part 87 of the same ligament 86. A saw blade from a modified or conventional bone cutting device is inserted into passages defined by lines 81 and 82 to create cut bone section 83. Bone Section 83 is separated from tibia 80 so that it is free to move in the direction of arrow 88. When bone section 83 is separated from tibial bone 80 the thickness of the saw cut adjacent lines 81 and 82 and the angle of cut will dictate the extent of travel of bone section 83 in the direction of arrow 88. This will in turn dictate the extent of overlap gained between cruciate ligament parts 84 and 85.us the thickness and angle of the cut will dictate the length of travel of the cut and the

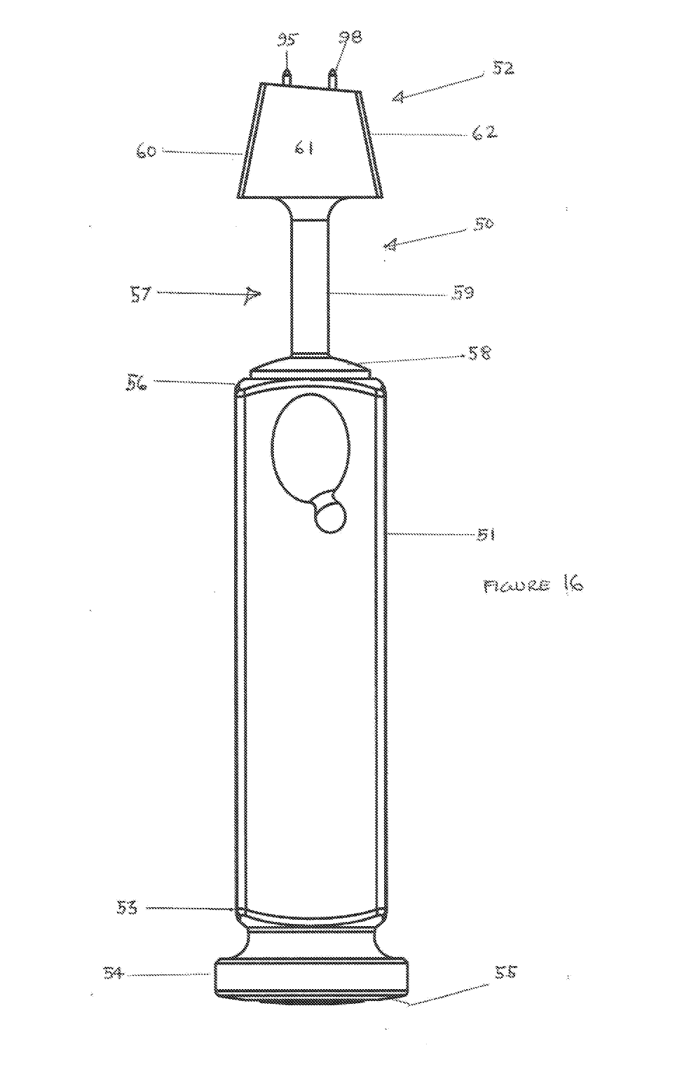

FIG. 14 shows a schematic view of the bone section 83 cut from tibial bone 80 and advanced to facilitate overlap of ruptured ends 84 and 85 of a cruciate ligament 86. In the embodiment shown bone section 83 is trapezoidal and has advanced a distance d from a pre cut position. Sides 90 and 91 of section 83 engage surfaces 92 and 93 respectively of tibia 80. Distance d will approximate the extent of overlap distance c between ruptured ligament parts 84 and 85. The cutting guide can allow various prismic shapes but the minimum width of the bone section will be dictated by the width of the footprint of the ruptured cruciate ligament. The smaller the angle of cut the greater the extent of travel of bone section 83. Likewise the larger the angle of cut the lower the distance of travel will be. The surgeon has flexibility to select from a kit of sizes of cutting guides the appropriate guide for a particular patent. Cruciate ligaments may vary in size from patient to patient requiring different cutting guide geometry. Also, in some cases only a small overlap of ruptured cruciate ligament will be required whereas in other a large overlap may be required depending upon how much cruciate is left after damage and the state of the ruptured ends.

FIG. 15 shows with corresponding numbering a perspective view of the tool 50 according to an alternative embodiment. As before handle 51 has a first end 53 which provides a platform 54 which allows support of the tool 50 and provides an under surface 55 which allows hammer impact when tool 50 is located in position. Guide element 57 includes a connector 58 which engages handle 51 and is detachable therefrom. Connecting stem 59 terminates at guide 52. Guide 52 according to the embodiment shown includes angled walls 60, 61, 62 and 63 which define in conjunction with platform base 64 an internal passage 65 which is angled in a similar manner to the walls 60, 61, 62 and 63. Extending from cutting guide 52, are four locating posts 95, 96, 97 and 98 which in use are hammered by a surgeon into tibial bone prior to cutting to secure guide 52 and prevent movement when bone cutting through passage 65. This anchors cutting guide attachment 57 against a bone surface to be cut and prevents once in situ, unwanted rotation of the tool 50 and cutting guide 52 during cutting and drilling. Posts 95, 96, 97 and 98 are located in the bone section 83 to be cut rather than outside the cut area as described previously in FIG. 13. Once the cut is completed the bone section 83 will require separation from posts 95, 96, 97 and 98 to allow bone section 83 freedom of movement. As before, tool 50 while anchored (in this case via posts 95, 96, 97 and 98) allows a surgeon to create a wedge or trapezoidal shaped bone section 83 which is free to move axially within a passage created by the cutting tool.

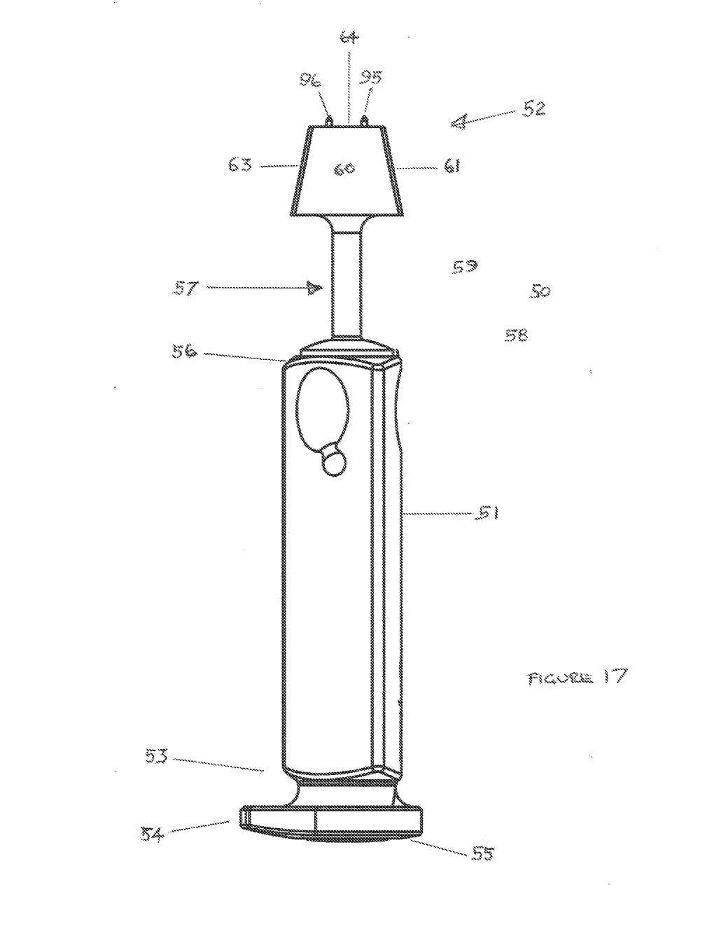

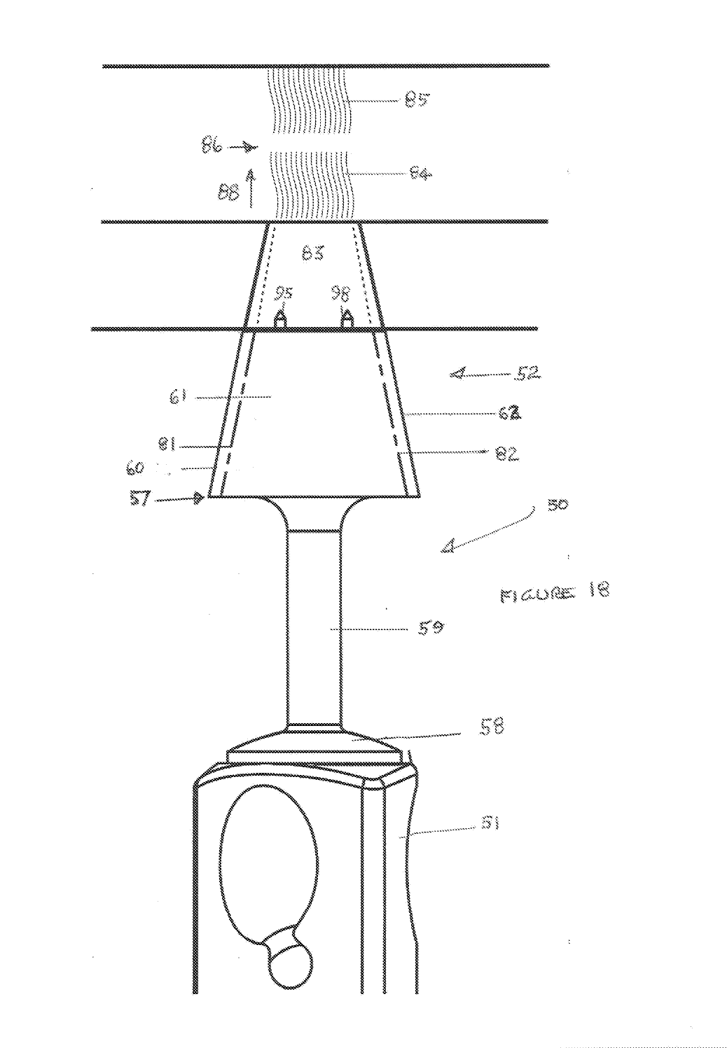

FIG. 16 shows with corresponding numbering a first side elevation view of the tool of FIG. 15. FIG. 17 shows with corresponding numbering a first side elevation view of the tool of FIG. 15.

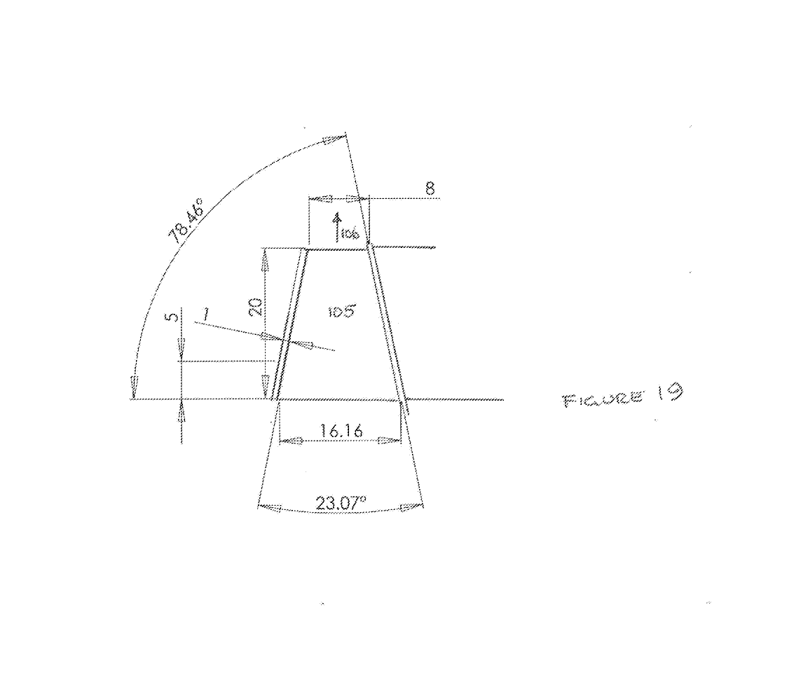

FIG. 18 shows with corresponding numbering an enlarged view of the working, end of the cutting guide 52 of FIG. 15 engaging a schematic view of tibial bone 80 via posts 95 and 98. Dotted lines 81 and 82 indicate a passage through which a saw blade may be passed advancing towards bone section 83. Extending from bone section 83 is a distal part 84 of a ruptured cruciate ligament 86 which opposes a proximal part 87 of the same ligament 86. A saw blade from a modified or conventional bone cutting device is inserted into passages defined by lines 81 and 82 to create cut bone section 83. As before bone Section 83 is separated from tibia 80 so that it is free to move in the direction of arrow 88. Prior to this, the surgeon separates bone section 83 from posts 95 and 98. When bone section 83 is separated from tibial bone 80 the thickness of the saw cut adjacent lines 81 and 82 and the angle of cut will dictate the extent of travel of bone section 83 in the direction of arrow 88. This will in turn dictate the extent of overlap gained between cruciate ligament parts 84 and 85. FIG. 14 shows a schematic view of the bone section 83 of FIG. 18 cut from tibial bone 80 and advanced to facilitate overlap of ruptured ends 84 and 85 of a cruciate ligament 86. In the embodiment shown bone section 83 is trapezoidal and has advanced a distance d from a pre cut position. Sides 90 and 91 of section 83 engage surfaces 92 and 93 respectively of tibia 80. Distance d will approximate the extent of overlap distance c between ruptured ligament parts 84 and 85. Once the overlap is effected, the surgeon can repair the cruciate. FIG. 19 shows according to one embodiment, the geometry, taper angle and dimensions of a cut bone section. The angle of cut according to this embodiment is 78.46 with a bone section maximum width 16.16 and a minimum width 8 mm. The gap between the tibial bone and the bone section cut is 1 mm. This geometry allows for an advancing distance of 5 mm. Trapezoidal wedge 105 can advance in the direction of arrow 106

The following description sets out a series of preferred but non limiting steps which a surgeon may adopt when using the tooling described herein to create a bone prism whose free movement over a selected distance enables repair of a ruptured anterior cruciate ligament (ACL). A standard C Guide is used to set an alignment between a ruptured ACL and an axis which will indicate a path for a guide wire. The guide wire traverses a path between the tibia and the ruptured ACL. This allows the surgeon to measure a distance a between an entry point in the tibia for the guide wire and the distal side of the ruptured ACL. A Computer guidance transmitter may be used to find an optimal angle of a block or wedge of bone to be drilled free of tibial bone. Distance a is a length between an entry point in the proximal tibia and a footprint centroid of ruptured ACL component anchored on the tibia. In a second step a guide wire is inserted between the entry point and the centroid of the ACL component. The guide wire left in situ may have 0.5 mm laser markings or a depth gauge can be used to measure distance a. The wire would have a known length. In a third step a slotted cutting block is urged against the tibial bone with its centre aligned with the path of the guide wire. Spikes are included on the plate spaced for centralizing the plate. For example four equally spaced spikes about 3 mm in length are provided. In a fourth step a cutting block is cut down on four sides of the block using a reciprocating saw using a 1 mm blade×5 mm.

Once a cut has been formed in the bone the cutting device is removed but the guide wire is kept in situ. The cut piece of bone which according to one embodiment is wedge shaped or trapezoidal bone section which advances proximally along the female passage preferably about 4 mm but within a range of 1-20 mm. When the bone section is advanced proximally, a transfixation wire is inserted transversely to lock the bone section from further movement relative to the tibia once ruptured ends of the cruciate ligament have been stitched together. Since the cut bone section is preferably wedge shaped—in that it has a wider distal end and a narrow proximal end. Since an inner wall of the tibia farm which the bone section has been removed, is also tapered, movement of the bone section in the proximal direction will cause wedging of the section after it has advanced about 4 mm within the available range of movement. The cutting tool can be selected to release a bone section which achieves a desired limit of travel within the through passage. The angle of an outer surface of the bone section will dictate the length of the travel within the passage formed. Selecting a cutting angle for the outer surface contour of the bone section will impact on the limit of travel. For example if a 2 cm block of bone cut from the tibia and having an apex of 8×6 mm, a taper angle of 27.5 would be required achieved by a 1 mm saw width and a 4 mm advancement to interlock the bone section in the wedge shaped or trapezoidal shaped channel. Likewise, if a 3 cm block of bone cut from the tibia and having an apex of 8×6 mm, a taper angle of 27.5 would be required and achieved by a 1 mm saw width and a 4 mm advancement to interlock the bone section in the wedge shaped or trapezoidal shaped channel. If a 2.5 cm block of bone cut from the tibia and having an apex of 8×6 mm, a taper angle of 27 would be required and achieved by a 1 mm saw width and a 4 mm advancement to interlock the bone section in the wedge shaped or trapezoidal shaped channel. An acute taper angle would be about 27 degrees.

A surgeon would select an appropriate cutting guide based on the cutting angle required. Guides are provided at different angles and cut widths to control the extent of axial travel of the bone section cut. In the case of a cruciate ligament the footprint on the tibial bone can vary from patient to patient with typical cruciate base sizes in the range of 8-12 mm. Sizes outside this range are also contemplated. As well as selection of the angle of cutting guide the thickness of cutting blades also impact on the extent of axial advancement of the bone section cut. The thicker the cutting blade the longer the travel distance. 1 mm wide cut may allow a 5-12 mm advance of the bone section. A preferred distance for advance of the bone section would be in the order of 5 mm at an angle of about 73-75 degrees. Also as bone has a certain elasticity this will also contribute to the overall extent of axial movement and can be allowed for in selection of cutting angles and thickness of cutting blades. Thus, it is proposed that a primary ACL repair using the research technique would bypass these intrinsic surgical difficulties as it does not require the surgeon to reproduce the ACL's complex multi strand spiral anatomy or ellipsoid attachment sites.

It will be appreciated by those skilled in the art that numerous variations and modifications may be made to the invention without departing from the overall spirit and scope of the invention broadly described herein.

1. A surgical tool for use in a ligament repair procedure after rupture of the ligament, the tool comprising:
a handle having a first end and a second end, the second end capable of receiving and retaining a cutting guide assembly;
the cutting guide including an outer wall which defines an internal space in which there is located a base;
a passage defined by the outer wall and base and which allows access for a cutting implement to bone; and at least one formation extending from the cutting guide which locates the tool against bone during cutting;
wherein the bone cutting guide, guides the cutting implement through said passage to enable cutting of a bone section which is separated from surrounding bone.

2. A tool according to claim 1 wherein the cut bone section is free to move relative to the surrounding bone in a passage in said bone created by the cutting tool.

3. A tool according to claim 2 wherein the passage in the guide is arranged so that a bone section cut from surrounding bone using the cutting tool has a wide end and a narrow opposite end.

4. A tool according to claim 3 wherein guide has a first end which in use engages a bone surface to be cut and a body which allows the bone section after cutting by the bone cutting tool to be wider at a distal end and narrower at a proximal end.

5. A tool according to claim 4 wherein the outer wall of the guide is inwardly tapered

6. A tool according to claim 5 wherein the outer wall of the cutting guide comprises a plurality of angled faces facing in different directions.

7. A tool according to claim 6 wherein the cutting guide is arranged to allow cutting of an inwardly tapered bone section

8. A tool according to claim 7 wherein the cutting guide is arranged to allow formation by cutting of a trapezoidal bone section.

9. A tool according to claim 8 wherein further comprising at least one anchorage post extending from the cutting guide which engage bone to prevent relative movement of the tool during bone cutting.

10. A tool according to claim 9 wherein wherein the cutting guide assembly is detachable from the handle.

11. A tool according to claim 10 wherein the first end includes a base which allows impact for insertion of the posts into bone.

12. A tool according to claim 11 wherein an axial opening through the cutting guide from the first to second end terminates in the base.

13. A tool according to claim 12 wherein the tool is capable of attachment to a drill.

14. A tool according to claim 13 wherein a pilot guide wire is insertable axially through the opening, in the cutting guide.

15. A tool according to claim 14 wherein there are four spaced apart anchorage posts extending from the wall of the cutting guide.

16. A tool according to claim 14 wherein there are four spaced apart anchorage posts extending; from the base of the cutting guide.

17. An assembly for use in a ligament reconstruction procedure after rupture of the ligament, the assembly comprising:
a jig capable of measurement of a length of a tibial bone section measured from a distal entry point to the tibia to a proximal location at which the length of bone is capable of separation form the tibia;
a guide wire for insertion axially through the hone section such that an entry point in the bone section aligns with a ruptured anterior cruciate ligament;
a bone cutting guide which has a first end which in use engages a tibial bone surface to be cut and a body which allows the bone section after cutting by the bone cutting element to be wider at a distal end than at a proximal end.

18. An assembly according to claim 17 wherein a bone cutting device forms a passage in said bone which the bone section is free to move therealong.

19. An assembly according to claim 18 wherein the bone cutting guide enables creation of a profile shape of the bone section which allows the bone section to advance a predetermined distance proximally within the passage.

20. An assembly according to claim 19 wherein the external contour of the cutting element allows an external contour of the bone section to limit proximal travel of the bone section over a distance within the range of 1-20 mm along the passage dictated by the contour.

21. An assembly according to claim 20 further comprising a drill which retains a drill bit having a first end which engages said cutting member and a second end which is retained by a drill.

22. An assembly according to claim 21 wherein the cutting member is capable of advancing and retracting inside a through passage in a guide element.

23. An assembly according to claim 22 wherein the bone cutting member is insertable in said through passage in the cutting guide element;

24. An assembly according to claim 23 further comprising a pilot wire insertable in a through passage in said cutting guide element;

25. An assembly according to claim 24 wherein the bone cutting guide element forms a passage in which the bone section is free to move therealong.

26. An assembly according to claim 25 wherein the passage created by the bone cutting guide element is tapered from wide to narrow in the proximal direction.

27. An assembly according to claim 26 wherein the assembly further comprises an extraction device which engages the cutting guide element to enable withdrawal of the cutting element at completion cutting.

28. An assembly according to claim 27 wherein the cutting guide element is shaped to allow the bone section to assume a wedge shape.

29. An assembly according to claim 28 wherein the cutting guide element is shaped to allow the bone section to assume a cone shape.

30. An assembly according to claim 29 wherein the cutting guide element is shaped to allow the bone section to assume a trapezoidal shape.

31. An assembly for use in the re attachment of a ruptured ligament, the assembly comprising;
a cutting tool operable by a power drill and having a first end terminating in a cutting profile and a second end attachable to the power drill, the cutting tool capable of creating by operation of the cutting profile a core of bone to which is attached part of a ligament requiring repair,
an instrument which receives a rod capable of entering a through passage in the instrument; the rod having a leading end which is capable of entering the cutting tool to engage the bone core to allow advancement and retraction of the bone core;
wherein the bone cutting tool has a first end which in use engages a tibial bone surface to be cut and a body which allows the bone section after cutting by the bone cutting tool to be wider at a distal end than at a proximal end.
